(19) United States
(12) Patent Application Publication
(10) Pub. No.: US 2022/0062057 A1
(43) Pub. Date: Mar. 3, 2022

(54) Pressure Applicator System and Method

(71) Applicant: Kristal Rutledge, Swatara, MN (US)

(72) Inventor: Kristal Rutledge, Swatara, MN (US)

(21) Appl. No.: 17/003,784

(22) Filed: Aug. 26, 2020

(51) Int. Cl. A61F 13/00

(52) U.S. Cl. CPC A61F 13/00051; A61F 2013/0028

(57) ABSTRACT

A pressure applicator system comprising a body including a covering member, a first wing, and a second wing, wherein the first wing and the second wing extend outwardly in a cantilever fashion from the covering member on opposite sides thereof; a strap including a first end attached to the first wing and a second end; and a fastening system for removably attaching the second end of the strap to the second wing, wherein the strap and body form a loop of adjustable size that may be placed about a desired location on a patient's body.

### FIELD OF THE DISCLOSURE

The invention relates generally to a pressure applicator, and more specifically to a pressure applicator removably positionable on a body part at a desired location to provide a desired pressure at the location.

### BACKGROUND

In many instances it is desirable to provide direct pressure at a particular location on the body. For example, applying direct pressure to a wound helps to prevent bleeding. Typically, a patch is applied to the wound that is held in place with adhesive tape applied to the patch and skin. Or, an adhesive bandage might be used. However, while such approaches might be sufficient to cover a wound, it does not allow for a desired pressure to be applied at the site.

Thus, there remains a need for an improved, simple, and cost-effective system and method for applying a desired amount of pressure at a particular site on a patient's body.

### SUMMARY OF THE INVENTION

The present disclosure provides a system and method for applying a desired amount of pressure at a particular desired site on a patient's body. In one embodiment, an applicator body includes a generally centrally disposed covering member adapted for placement at the desired site. Coupled to the covering member on opposite sides may be a first wing and a second wing. The first wing and second wing may act as flexible cantilevers extending outwardly from the covering member. A strap includes a first end attached to the end of the first wing opposite the covering member. The strap includes a second end which may be removably fastened to the end of the second wing opposite the covering member. Fastening means for removably coupling the second end to the second wing may comprise a hook and loop fastening system, a hook and nubs fastening system, a post and hole fastening system, a snaps fastening system, self-adhesive strips fastening system, etc. In that way, the covering member may be held at a desired site and apply a desired amount of pressure to the site by removably placing the strap about the body part proximate the site (e.g., around a leg, an arm, the head, etc.).

Other benefits and advantages of the present disclosure will be appreciated from the following detailed description.

### DESCRIPTION OF THE DRAWINGS

FIG. 1 is a perspective view of an exemplary embodiment of a pressure applicator system in accordance with the present disclosure.

FIG. 2 is a perspective view of the pressure applicator system shown in FIG. 1, with an end of the strap detached from the applicator body.

FIG. 3 is a front view of the pressure applicator system shown in FIG. 1.

FIG. 4 is a front view of the pressure applicator system shown in FIG. 2.

FIG. 5 is a top view of the pressure applicator system shown in FIG. 1.

FIG. 6 is a bottom view of the pressure applicator system shown in FIG. 1.

FIG. 7 is a front view of an alternate exemplary embodiment of a pressure applicator system in a fastened position in accordance with the present disclosure.

FIG. 8 is a front view of the alternate embodiment shown in FIG. 7, in an unfastened position.

FIG. 9a is an exploded perspective view of the pressure applicator system shown in FIG. 2, positioned about a patient's arm.

FIG. 9b is a perspective view of the pressure applicator system shown in FIG. 2, for fastening about the arm of a patient.

FIG. 9c is a perspective view of the pressure applicator system shown in FIG. 1, positioned on a patient's arm.

### DETAILED DESCRIPTION

Embodiments of the invention and various alternatives are described. Those skilled in the art will recognize, given the teachings herein, that numerous alternatives and equivalents exist which do not depart from the invention. It is therefore intended that the invention not be limited by the description set forth herein or below.

One or more specific embodiments of the system and method will be described below. These described embodiments are only exemplary of the present disclosure. Additionally, in an effort to provide a concise description of these exemplary embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Further, for clarity and convenience only, and without limitation, the disclosure (including the drawings) sets forth exemplary representations of only certain aspects of events and/or circumstances related to this disclosure. Those skilled in the art will recognize, given the teachings herein, additional such aspects, events and/or circumstances related to this disclosure, e.g., additional elements of the devices described; events occurring related to pressure application; etc. Such aspects related to this disclosure do not depart from the invention, and it is therefore intended that the invention not be limited by the certain aspects set forth of the events and circumstances related to this disclosure.

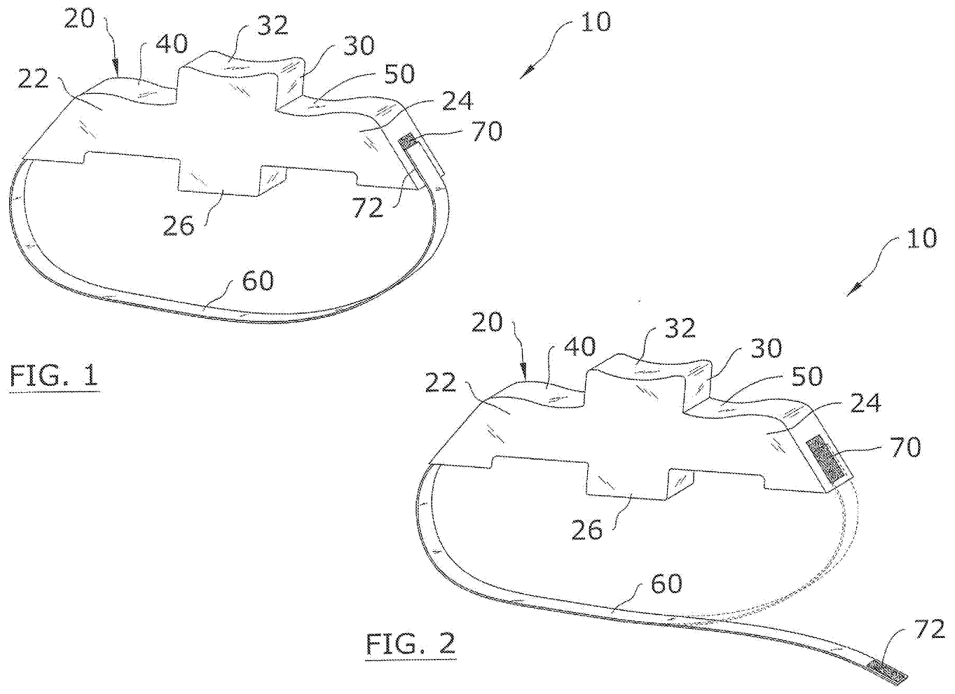

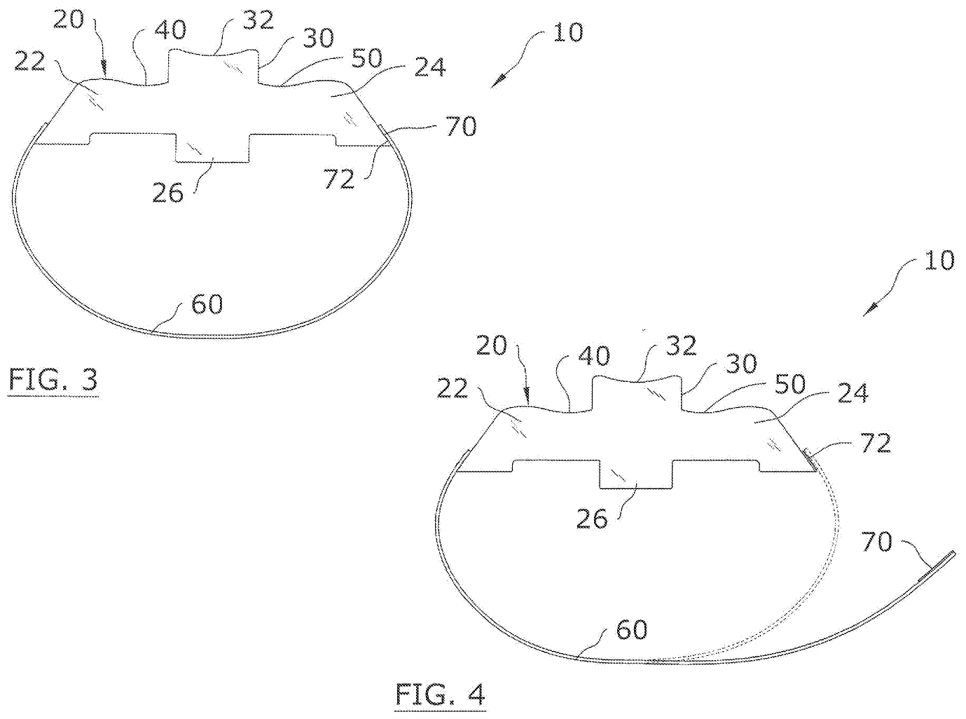

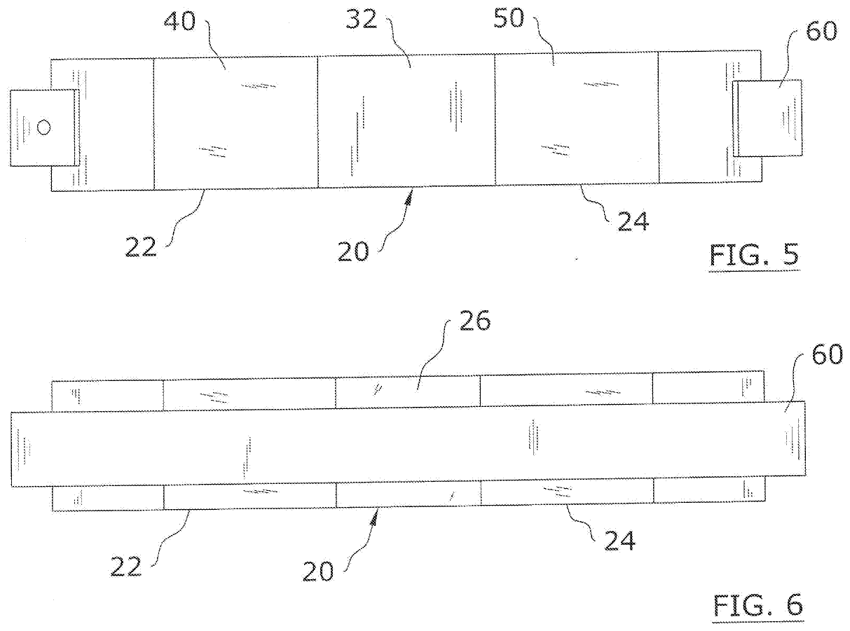

Turning now to the drawings, in which similar reference characters denote similar elements throughout the several views, an exemplary embodiment of a pressure applicator system 10 is shown in FIGS. 1-6 and 9a-9c. A body 20 includes a covering member 26, and a first wing 22 and a second wing 24 disposed on opposite sides of the covering member 26 and extending outwardly therefrom. A strap 60 includes a first end attached to first wing 22 and a second end removably attached to the second wing 24. In one embodiment, the second end of strap 60 includes a hook 70 and loop 72 fastening system for removably securing the second end of strap 60 in place a second wing 24.

In one embodiment, the upper surface of body 20 includes one or more depressions 32, 40, 50 each adapted to receive one or more fingers or other portions of a caregiver' s hand during positioning of the body 20, e.g., on a patient's arm. In one embodiment, a portion of the covering member 26 extends downwardly from the bottom surfaces of first wing 22 and second wing 24. In this way, the bottom surface of covering member 26 may be more easily positioned atop a desired location, e.g., a wound. In one embodiment, a medical pad 16 may be positioned between the bottom surface of covering member 26 and a patient's wound 14.

In one embodiment, the first wing 22 and the second wing 24 may act a flexible cantilevers extending outwardly from covering member 26. In that way, during positioning of the body 20 (e.g., about an arm 12) the lower end of body 20 may better conform to the surface upon which body 20 is applied.

As illustrated, the strap 60 may be removably secured as desired to second wing 24 so that a desired amount of pressure may be applied by the lower surface of covering member 26, e.g., onto a wound or other desired location.

The body 20 may be constructed of either reusable or disposable materials. In some embodiments, the body 20 may be formed of plastic, paper, cardboard, or other materials, either alone or in combination. In one embodiment, the lower surface of covering member 26 may include a medical pad, gauze, bandage, or other dressing that may be applied directly to a wound or other desired location.

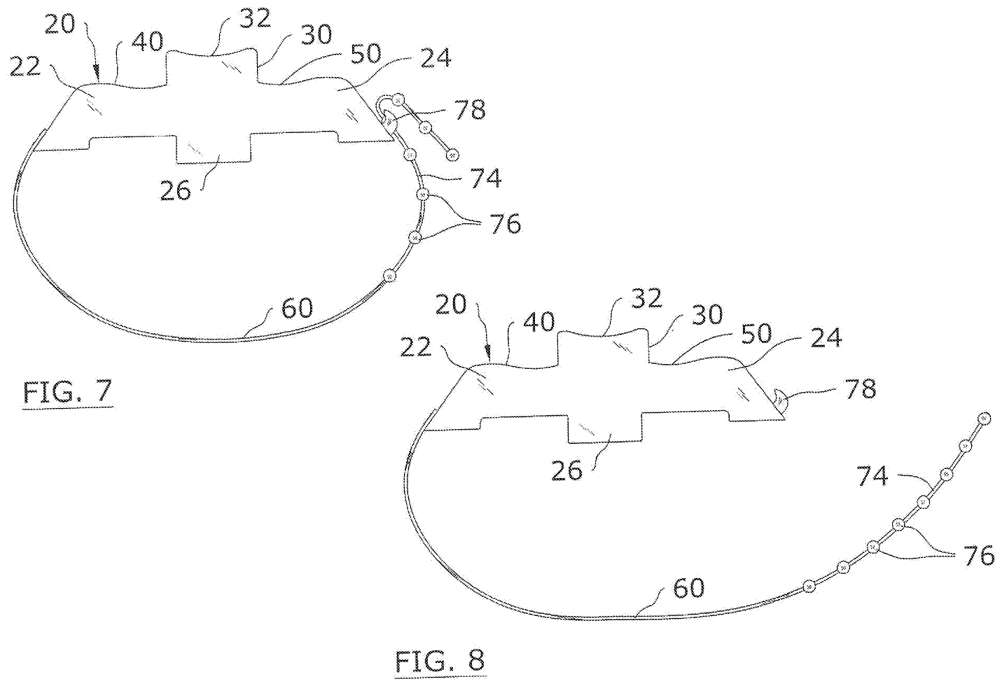

FIGS. 7 and 8 illustrate an exemplary embodiment of an alternate pressure applicator system that includes an alternate form of a fastening device. As shown in FIGS. 7-8, the second end of strap 60 may include a plurality of nubs 76 with a space 74 therebetween. The surface of second wing 24 may include a hook 78. Securing the pressure applicator in place thus involves positioning a desired nub 76 at the hook 78, so as to deliver a desired amount of pressure via covering member 26.

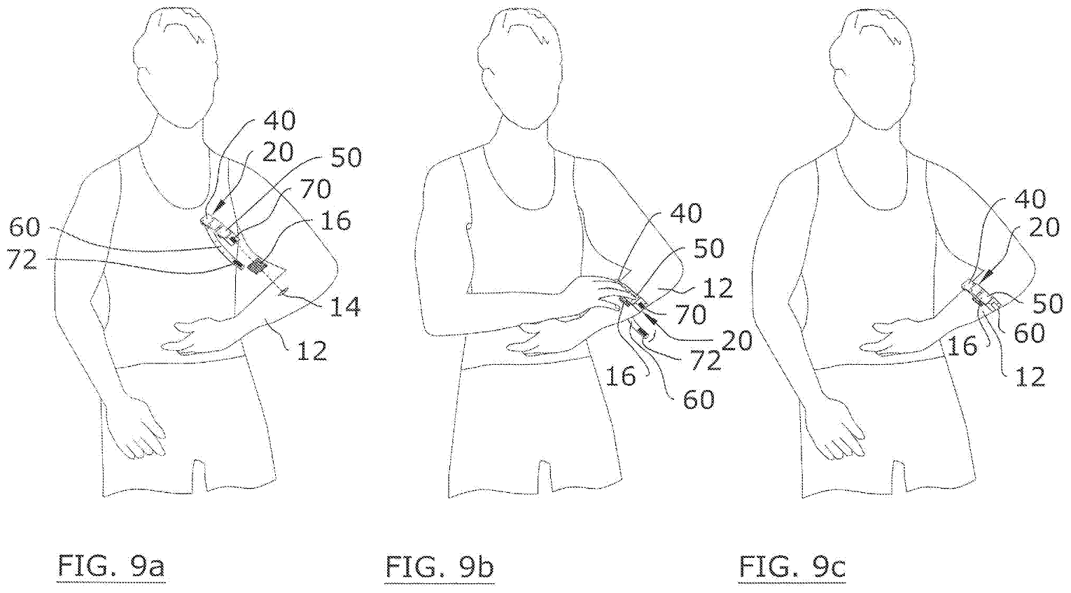

As shown in FIGS. 9a-9c, an exemplary pressure applicator in accordance with the disclosure may be positioned about an individual's arm 12. However, the pressure applicator may be secured about other body locations and be accordingly sized therefor. Also, the device may be used at a wound or another desired location where pressure is to be applied (e.g., a needle access site, a dialysis access site, etc.).

The fastening system of the pressure applicator may include a hook and loop fastening system, a nub and hook fastening system, a snaps fastening system, a post and hole fastening system, or a self-adhesive strip fastening system.

In an exemplary use, a user first positions a medical pad 16 upon a wound 14 and then positions the body 20 so that covering member 26 is in contact with the medical pad 16. The user then applies a compression force upon the body 20 by positioning fingers or other parts of a hand within the depressions 32, 40, 50 of the body 20. When the desired amount of compression force is applied, the user then wraps the strap 60 around the body part of the patient and secures the strap 60 to the second wing 24 in a desired location to maintain a desired level of compression force upon the medical pad 16 and wound 14, e.g., to promote coagulation. The strap 60 about the body part may be adjusted in length simply by detaching the second end of strap 60 from second wing 24 and reattaching the second end to make more or less of a loop of strap 60. This might be done, for example, at any time the patient becomes uncomfortable, or when it is apparent that more or less compression force is required. When desired (e.g., when the wound 14 has been coagulated), the second end of strap 60 may be removed from second wing 24 and the body 20 removed from the medical pad 16 and wound 14, and the pressure applicator may be discarded appropriately.

It should be understood that the foregoing description is only illustrative of the invention. Various alternatives and modifications can be devised by those skilled in the art having the benefit of this disclosure, without departing from the invention. Accordingly, the invention is intended to embrace all such alternatives, modifications and variances.

Certain exemplary embodiments of the disclosure may be described. Of course, the embodiments may be modified in form and content, and are not exhaustive, i.e., additional aspects of the disclosure, as well as additional embodiments, will be understood and may be set forth in view of the description herein. Further, while the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention.

What is claimed is:

1. A pressure applicator system comprising:
a body including a cover, a first wing, and a second wing, wherein the cover, first wing, and second wing are integrally formed, and wherein the first wing and the second wing extend outwardly in a cantilever fashion from the cover on opposite sides thereof;
a strap including a first end attached to the first wing and a second end; and
a fastener removably attaching the second end of the strap to the second wing,
wherein the strap and body form a loop of adjustable size configured to be placed about a desired location on a patient's body.

2. The pressure applicator system of claim 1, wherein the cover is configured to contact a desired site on a patient's body and the loop is adjusted so that the cover is configured to deliver a desired pressure against the desired site.

3. The pressure applicator system of claim 1, wherein the fastener is a hook and loop fastener.

4. The pressure applicator system of claim 1, wherein one or both of the first wing and the second wing are flexible.

5. The pressure applicator system of claim 1, wherein the body and strap are disposable.

6. The pressure applicator system of claim 1, wherein an upper surface of the body includes one or more depressed surfaces adapted for receiving a portion of a user's hand.
